(19) United States
(12) Patent Application Publication
(10) Pub. No.: US 2024/0173076 A1
(43) Pub. Date: May 30, 2024

(54) SAFETY SENSOR FOR LASER INDUCED PRESSURE WAVE IN CONTRAST MEDIA WITHIN A CONTAINMENT CATHETER DURING VASCULAR THERAPY

(71) Applicant: KONINKLIJKE PHILIPS N.V., EINDHOVEN (NL)

(72) Inventor: Tyler West CRONE, COLORADO SPRINGS, CO (US)

(21) Appl. No.: 18/284,090

(22) PCT Filed: Mar. 25, 2022
(86) PCT No.: PCT/EP2022/057857

§ 371 Date: Sep. 26, 2023

Related U.S. Application Data
(60) Provisional application No. 63/167,667, filed on Mar. 30, 2021.

(51) Int. Cl. A61B 18/24; A61B 17/00; A61B 18/00; A61M 5/00; A61M 25/01

(52) U.S. Cl. CPC A61B 18/245; A61M 5/007; A61M 25/0105; A61B 2017/00057; A61B 2017/00079; A61B 2018/00345; A61B 2018/00577; A61B 2018/00666; A61B 2018/00773; A61B 2560/0266

(57) ABSTRACT

A vascular therapy device (10) includes a catheter (12) including at least one optical fiber (18) with a light output aperture (20). A containment sheath (14) has a sheath opening (13) disposed at an end thereof. The catheter is disposed inside the containment sheath and movable relative to the containment sheath to extend the light output aperture beyond the sheath opening such that the light output aperture is outside of the containment sheath. A safety sensor (22) is configured to detect whether the light output aperture is in a safe zone inside the containment sheath.

### FIELD

The following relates generally to the catheter arts, thrombectomy arts, atherectomy arts, catheter safety arts, and related arts.

### BACKGROUND

Vascular therapy (e.g., thrombectomy, atherectomy, and so forth) devices are medical devices designed to remove or modify tissue or material from inside a diseased vessel (e.g., an artery, a vein, etc.). Intravascular devices attempt to remove this material without surgically opening the vessel. The mechanisms of intravascular removal can include simple suction, mechanical cutting, chemical dissolution, ablation through heat or light, maceration by mechanical or sonic energy, and so forth.

The mechanism of action in, for example, some types of laser atherectomy utilizing a laser for material removal, is referred to as ultraviolet (UV) photoablation. However, this approach is less effective for calcified deposits, due to lower absorption of the laser light (e.g., in the near ultraviolet, such as at 308 nm in some specific systems). The photoablation can be enhanced for the purposes of debulking tissues at the target sites, especially but not limited to calcified deposits. The use of contrast media in combination with a UV laser produces a Laser Induced Pressure Wave (LIPW). This involves laser energy absorption at a molecular level in the contrast media, release of that energy producing a vapor bubble which expands, followed by a rapid collapse. This mechanism is effective for the purpose of debulking plaque and/or calcified lesions within the blood vessel. In order to do this safely, the lasing in contrast protocol is performed with the laser and proximate absorbing contrast medium located within a containment catheter.

The following discloses certain improvements.

### SUMMARY

In some embodiments disclosed herein, a vascular therapy device includes a catheter including at least one optical fiber with a light output aperture. A containment sheath has a sheath opening disposed at an end thereof. The catheter is disposed inside the containment sheath and movable relative to the containment sheath to extend the light output aperture beyond the sheath opening such that the light output aperture is outside of the containment sheath. A safety sensor is configured to detect whether the light output aperture is in a safe zone inside the containment sheath.

In some embodiments disclosed herein, a vascular therapy device includes a catheter including at least one optical fiber with a light output aperture. A containment sheath has a sheath opening disposed at an end thereof. The catheter is disposed inside the containment sheath and movable relative to the containment sheath to extend the light output aperture beyond the sheath opening such that the light output aperture is outside of the containment sheath. A proximity sensor is configured to detect passage events in which the light output aperture passes through the sheath opening. An electronic processor is configured to detect whether the light output aperture is in the safe zone based at least on the passage events detected by the proximity sensor.

In some embodiments disclosed herein, a vascular therapy device includes a catheter including at least one optical fiber with a light output aperture. A containment sheath has a sheath opening disposed at an end thereof. The catheter is disposed inside the containment sheath and movable relative to the containment sheath to extend the light output aperture beyond the sheath opening such that the light output aperture is outside of the containment sheath. A safety sensor is configured to detect whether the light output aperture is in a safe zone inside the containment sheath. The device is configured to operate in a saline mode in which saline flows through the containment sheath and in a contrast mode in which contrast medium flows through the containment sheath. The device further includes a device controller configured to automatically turn off a laser operating to inject light into the at least one optical fiber of the catheter in response to a safety interlock condition in which the device is operating in contrast mode and the safety sensor detects that the light output aperture is not in the safe zone inside the containment sheath.

One advantage resides in providing an intravascular device with a safeguard feature to prevent blood vessel damage.

Another advantage resides in providing an intravascular device with a safety sensor to prevent blood vessel damage.

Another advantage resides in providing an intravascular device with one or more sensors to determine when an ablating instrument operating under conditions that generate LIPW is within a containment sheath.

Another advantage resides in providing an intravascular device with one or more sensors to determine when an ablating instrument is within a containment sheath and a controller to cease supply of energy to an ablating instrument when the ablating instrument is outside of the containment sheath and is operating under conditions that generate LIPW.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a vascular therapy device in accordance with the present disclosure.

FIG. 2 diagrammatically illustrates another view of the device of FIG. 1.

FIG. 3 diagrammatically illustrates another view of the device of FIG. 1.

FIG. 4 diagrammatically illustrates another view of the device of FIG. 1.

FIG. 5 diagrammatically illustrates another embodiment of the device of FIG. 1.

FIG. 6 diagrammatically illustrates a method of performing a vascular therapy method using one of the devices of either FIG. 1 or FIG. 5.

### DETAILED DESCRIPTION

The following relates to an improved thrombectomy or atherectomy device. Such devices are used to remove atherosclerosis (i.e., plaque buildup) in an artery (atherectomy procedure) or to remove a thrombus or collagen buildup in a vein (thrombectomy procedure). These procedures are typically performed in the peripheral vascular system to treat blood vessels in a leg or arm, although the procedures may be applied to treat blood vessels in other anatomical areas.

The following more specifically relates to an improvement in a system for laser atherectomy or thrombectomy. This system includes a sheath (e.g., containment catheter; also referred to herein as a containment sheath) which surrounds an inner catheter (also called a laser catheter herein) that includes an optical fiber or fiber bundle with a light output aperture. In one typical workflow, in a first pass, the light output aperture is extended out of an open end of the containment catheter and a laser beam that is input to the optical fiber or fiber bundle directly ablates the lesion. This is usually done with saline flowing through the sheath, in what is referred to as a saline mode or saline infusion protocol.

However, the laser light is not strongly absorbed by calcium deposits, so that this first pass does not effectively remove calcium deposits. To address this problem, a second pass is performed as follows. The light output aperture is withdrawn back into the containment catheter, and the flow is switched from saline to a contrast medium, thus placing the device into contrast mode or a contrast infusion protocol. The laser beam is strongly absorbed by the contrast medium, breaking molecular bonds of the contrast medium, which releases the absorbed energy by producing an acoustic or sonic wave known as the Laser Induced Pressure Wave (LIPW) and resulting in the formation of a cavitation vapor bubbles. The LIPW passes through the containment catheter and interacts with calcium deposits to debulk the calcified plaque. It should be noted that this two-pass workflow is typical, other workflow sequences may be used. For example, the operator may switch back and forth between saline and contrast modes to gradually remove difficult deposits.

It is recognized herein that there may be a potential safety hazard with operation in the contrast mode, that is, under conditions that generate LIPW. Since the contrast medium flows out of the open end of the containment catheter, if the laser is operated when the light output aperture is extended out of the open end of the containment catheter then the contrast medium outside of the containment catheter absorbs the laser beam and LTPW is generated outside of the containment catheter, which can damage the blood vessel wall.

To address this problem, a safety sensor is provided as disclosed herein to detect when the light output aperture is extended outside of the containment catheter, and the system controller is configured to use information from the safety sensor to implement a safety interlock that prevents operation under conditions that generate LIPW when the light output aperture is not in a safe zone within the containment sheath. In the illustrative embodiment, the safety sensor includes a sheath end marker disposed on the containment catheter (i.e., sheath) at or near the open end of the containment catheter, and an aperture marker disposed on the laser catheter at or near the light output aperture. One of these markers is an active sensor which detects proximity of the other marker. Optionally, an LIPW zone marker is also disposed on the containment catheter at a location marking the boundary of the target LIPW operating zone within the containment catheter.

The controller is configured (e.g., by operative connection to receive readings from the safety sensor and by suitable programming) to implement the safety interlock feature, for example as follows. A state is maintained, which indicates whether the light output aperture is in within the containment catheter. The state is initial set to “inside” since the light output aperture is usually inside the containment catheter while the catheter is inserted into the vasculature and moved to the treatment site, and changes to “outside” in response the aperture marker crossing the sheath end marker during the start of the first pass in saline mode. Thereafter, the state changes back to “inside” in response to the aperture marker again crossing the sheath end marker during the retraction of the light output aperture back inside the containment catheter. The controller automatically turns off the laser if both of the following conditions are met: (1) the operating mode is contrast mode (e.g., suitably defined as any time the contrast medium is flowing) and (2) the state is “outside”.

If the LIPW zone marker is also provided, then a zone state is similarly maintained which indicates whether the light output aperture is extended beyond the LIPW zone marker (“inside” zone) or further withdrawn into the containment catheter (“outside” zone). In this case, the laser is automatically turned off if: (1) the operating mode is contrast mode and (2) either the state is “outside” or the zone state is “outside”. (In a variant embodiment, since the laser typically should not be used at all when the light output aperture is withdrawn further in than the safe zone for LIPW operation, the laser may be automatically turned off any time the state indicates the light output aperture is withdrawn further in than the safe zone).

The illustrative safety sensor employing one or two proximity sensors with components disposed on the laser catheter and containment sheath has advantages such as providing a compact design fully integrated with the device, and providing for relatively straightforward state-based data analysis to implement the safety interlock. However, other types of safety sensors are contemplated for monitoring the state (and optionally also the zone state), such as deriving the location of the light output aperture relative to the sheath opening from X-ray fluoroscopy images acquired during the laser atherectomy procedure (which is typically performed as an image-guided therapy (IGT)). Also, while laser atherectomy is typically performed in an artery, the disclosed devices and approaches are also suitably used in venous therapies, e.g., a thrombectomy.

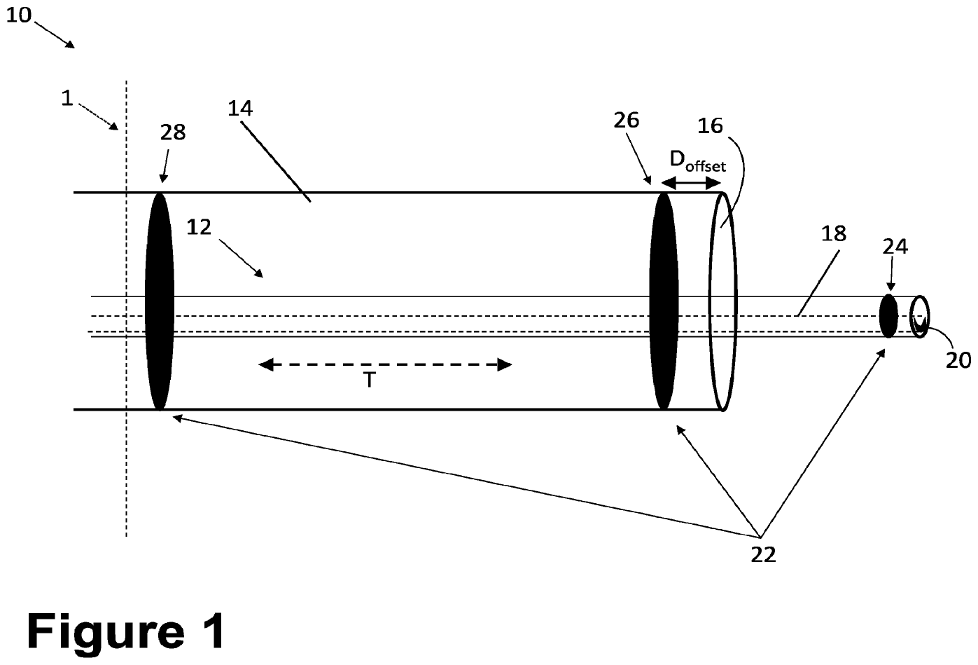

With reference to FIG. 1, an illustrative vascular therapy (i.e., thrombectomy or atherectomy) device 10 is diagrammatically shown. The device 10 can be any suitable vascular therapy device (e.g., a Thor vascular therapy device, available from Koninklijke Philips N.V., Eindhoven, the Netherlands). As shown in FIG. 1, the device 10 includes a catheter 12 surrounded by a containment catheter (also referred to herein as a containment sheath, or simply as a sheath) 14. The illustrative containment catheter 14 surrounds the catheter 12 (i.e., the catheter 12 is an inner catheter relative to the containment sheath 14). The catheter 12 is movable at least along a longitudinal translation direction T denoted in FIG. 1 within the containment sheath 14. In some embodiments, a distance between an outer diameter of the catheter 12 and an inner diameter of the containment sheath 14 can be, for example, 0.0021 inches, thereby providing a channel between the catheter 12 and the containment sheath 14 through which saline and/or a contrast medium can flow.

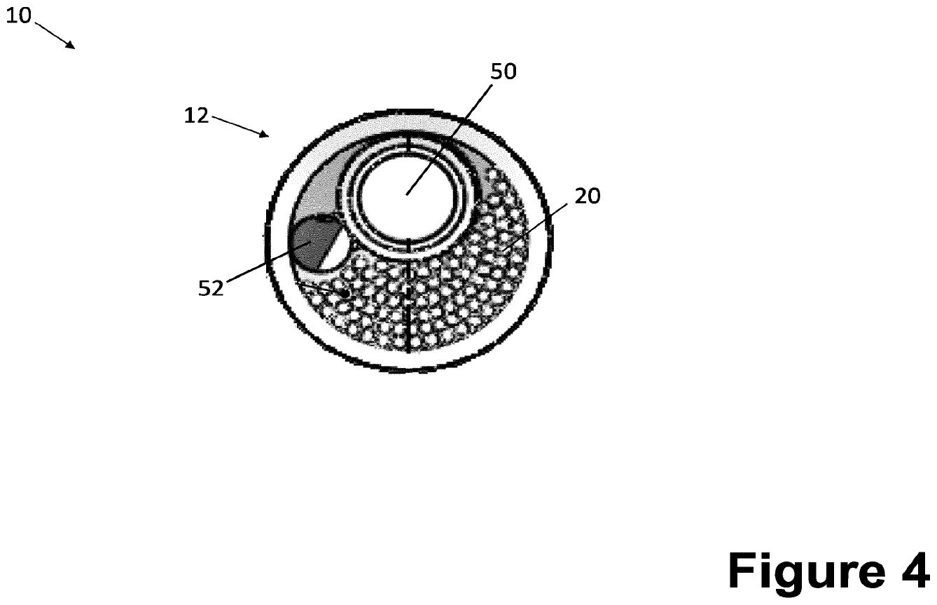

At one end of the containment sheath 14 is a sheath opening 16 through which the distal end of the catheter 12 can move into and out of the containment sheath 14. The catheter 12 includes at least one optical fiber 18 (shown using dashed lines) with a light output aperture 20 that together comprise a tip of the catheter 12. The illustrative light output aperture 20 is eccentrically positioned relative to the center axis of the catheter 12, and is located at the very end of the catheter 12. However, in other embodiments the light output aperture could have different geometry, e.g., could be centered on the center axis of the catheter 12 and/or could be located on a sidewall of the catheter 12 by way of a suitable bend in the optical fiber 18. It will be appreciated that the optical fiber 18 may in some embodiments comprise an optical fiber bundle, as shown for example in FIG. 4. The optical fiber(s) 18 are configured to carry light energy of a wavelength and intensity effective to perform UV photoablation or otherwise remove (i.e., ablate) a blood clot (e.g., a thrombus), plaque (e.g., atheroma), or other undesired material disposed on a blood vessel wall, by way of direct exposure of the material to the light (e.g., in saline mode) or by way of generating LIPW energy (e.g., in contrast mode), depending on the operational mode. The catheter 12 is disposed inside the containment sheath 14 and movable (at least movable back and forth along the translation direction T) relative to the containment sheath 14 to extend the light output aperture 20 beyond the sheath opening 18 such that the light output aperture 20 is outside of the containment sheath 14.

FIG. 1 also shows a safety sensor 22 configured to detect whether the light output aperture 20 is in a safe zone inside the containment sheath 14 (the safe zone is defined by lines 1 and 2 as shown in FIG. 1). The safety sensor 22 is disposed on an end portion of the containment sheath 14; although the safety sensor 22 may be disposed on any suitable portion of the containment sheath 10. In some embodiments, the safety sensor 22 comprises a proximity sensor configured to detect passage events in which the light output aperture 20 passes through the sheath opening 16. The passage events can include, for example, whether the light output aperture 20 has extended beyond the sheath opening 18, whether the light output aperture 20 (once detected past the sheath opening 18) has been withdrawn into the containment sheath 14, and so forth. The proximity sensor 22 can be any suitable sensor to implement suitable detection methods, such as capacitive coupling, induction, infrared, photo-detection, ultrasonic, Hall effects, and so forth.

As shown in FIG. 1, the proximity sensor comprises an aperture marker 24 disposed on the catheter 12, and a sheath opening marker 26 disposed on a portion of the containment sheath 14. One of the aperture marker 24 and the sheath opening marker 26 is a passive marker, and the other of the aperture marker 24 and the sheath opening marker 26 is an active marker configured to detect proximity of the passive marker. For example, in one embodiment the aperture marker 24 is a passive marker, and the sheath opening marker 26 is an active marker configured to detect proximity of the passive aperture marker. In another embodiment, the sheath opening marker 26 is a passive marker, and the aperture marker 24 is an active marker configured to detect proximity of the passive sheath opening marker. The passage events detected by the markers 24, 26 can include whether the aperture marker 24 has extended beyond the sheath opening marker 26.

In some embodiments, as shown in FIG. 1, the safety sensor 22 further includes a backside proximity sensor 28 configured to detect passage events in which the light output aperture 20 passes through an interior boundary of the safe zone of the containment sheath 14. The interior boundary is located inside the containment sheath 14 and defined by line 1.

The sheath opening marker 26 is preferably located close to the sheath opening 16. In some embodiments, the sheath opening marker 26 is interior of the sheath opening 16 by a longitudinal offset distance D_{offset} that provides a safety margin chosen to account for the distance over which light exiting the light output aperture 20 may induce LIPW in the contrast medium. In some embodiments, the working distance of the light exiting the light output aperture 20 is small, e.g., on the order of microns or tens of microns, in which case the offset distance D_{offset} can be small or even zero (i.e., the marker 26 could be precisely at the sheath opening 16).

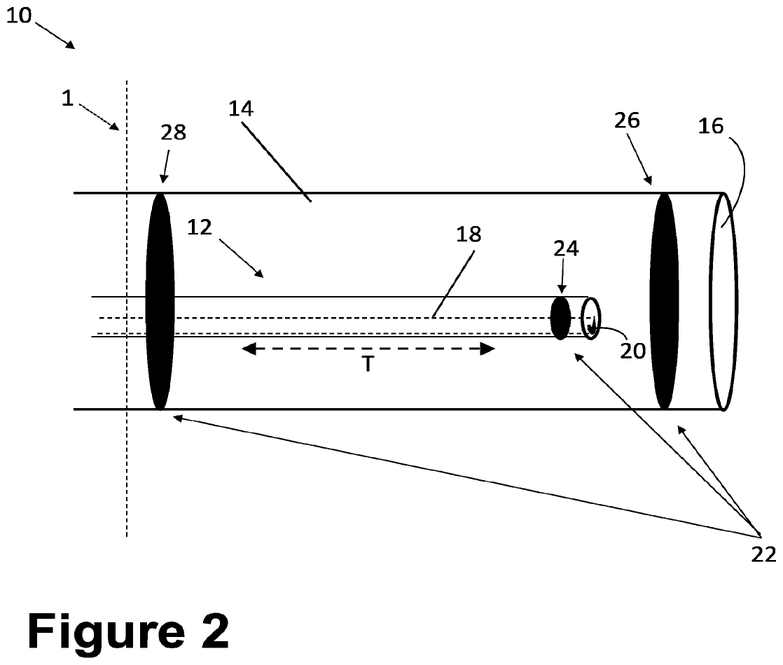

FIG. 1 shows the light outlet aperture 20 and the aperture marker 24 outside of the sheath opening 18. FIG. 2 shows the light outlet aperture 20 and the aperture marker 24 inside of the sheath opening 18.

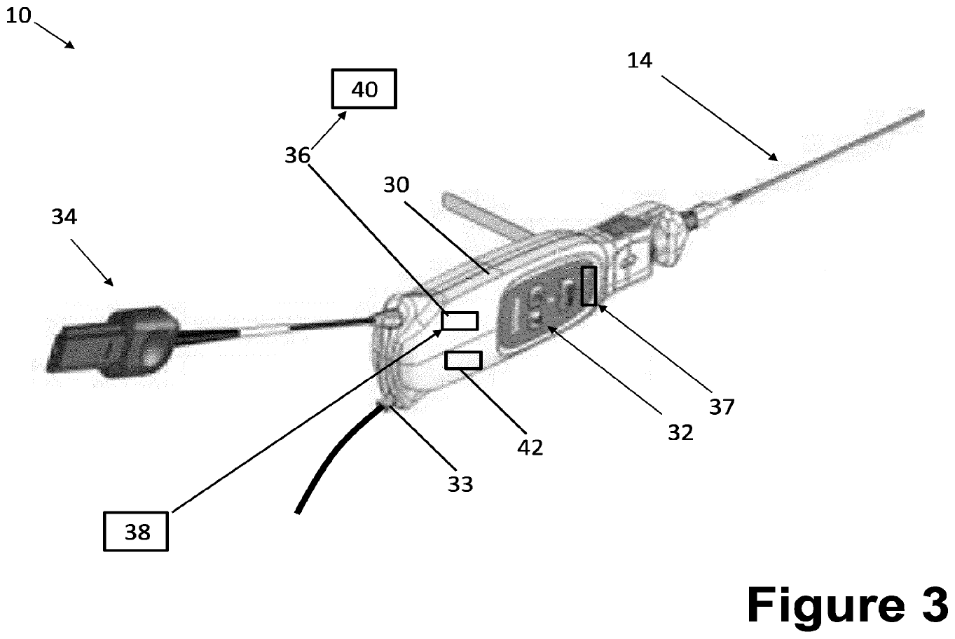

With reference now to FIG. 3, and with continuing reference to FIGS. 1 and 2, the containment sheath 14 and the catheter 12 (not shown in FIG. 3 but is disposed within the containment catheter 14 shown in FIG. 3) are connected with a housing 30 of the device 10. The device 10 also includes a motor drive unit (MDU) 32 configured to drive movement of the catheter 12 within a target area (e.g., an artery or a vein). The device 10 further includes a laser coupler 34 operatively connected with the at least one optical fiber 18 and an energy supply (not shown) to supply energy to the optical fiber(s) 18 to deliver ablation therapy to a lesion in the artery or vein. A fluid inlet 33 enables flow of a working fluid into the containment sheath 14. For example, the working fluid may be switched between saline which is flowed when operating in saline mode, and contrast medium which is flowed when operating in contrast mode. The contrast medium should be absorbing for the light generated by the laser so that the contrast medium is energized to produce LIPW. Optionally, the contrast medium may also be absorbing for X-rays used in fluoroscopic imaging optionally performed to monitor the interventional vascular treatment procedure.

Within the housing 30 is an electronic processor 36 (e.g., a microprocessor) configured to control operation of the MDU 32 and/or the laser coupler 34. In some examples, the electronic processor 36 can be an external computing device in electronic communication with the device 10. The electronic processor 36 is configured or programmed to detect whether the light output aperture 20 is in the safe zone based at least on the passage events detected by the proximity sensor 22. In some examples, when the device 10 includes the backside proximity sensor 28, the electronic processor 34 is configured to detect whether the light output aperture 20 is in the safe zone further based on the passage events detected by the backside proximity sensor 28. In some examples, a display device 37 can be included in the housing 30 and be operatively connected with the electronic processor 36. The display device 37 can display information related to the ablation process, as described in more detail below. The electronic processor 36 also has information on whether the device is operating in contrast mode. In one approach, a user input (e.g., a switch) is used to enable the user to manually identify to the electronic processor 36 whether saline or contrast medium is flowing into the inlet 33. In another approach, an optical sensor can be provided inside the housing 30 to automatically detect when contrast medium is flowing. For example, the optical sensor can be implemented as an LED/photodetector arrangement that detects the opacity of the fluid, with contrast medium being detected due to its higher opacity. In yet another approach, separate inlets are provided for the saline and contrast medium, and the housing 30 includes valves for switching between saline mode and contrast mode, and the electronic processor 36 controls those valves and hence the mode information is available to the electronic processor.

The electronic processor 36 can be configured or programmed to detect whether the light output aperture 20 is within the safe zone shown in FIG. 1. In one example embodiment, one or more of the aperture marker 24 and the sheath opening marker 26 can be radiopaque. The electronic processor 36 is then configured to receive images from an interventional radiology imaging device 38 (shown diagrammatically in FIG. 1 as a rectangle), such as a live fluoroscope, that depict the radiopaque aperture marker 24 and the radiopaque sheath opening marker 26. In this embodiment, the radiopaque aperture marker 24 and the radiopaque sheath opening marker 26 do not detect passage events, and thus are separate from the safety or proximity sensor 22. In this embodiment, the fluoroscope 38 comprises the safety sensor 22. The electronic processor 36 then detects, from the received images, whether the light output aperture 20 is in the safe zone. (In another approach, an electronic processor of the fluoroscope 38 performs the image processing to detect whether the light output aperture 20 is in the safe zone, and sends an indication of whether the light output aperture 20 is in the safe zone to the processor 36 of the catheter MDU 30). In some embodiments, the safe zone does not have an interior boundary within the containment sheath 14 (e.g., as defined in FIG. 1 by line 1). In such embodiments, an entire inside of the containment sheath 14 (or at least the entire portion of the inside of the containment sheath or at least the entire portion of the inside of the containment sheath 14 within which the light output aperture 20 could be located within which the light output aperture 20 could be located) is defined as the safe zone.

In another example embodiment, the electronic processor 36 is programmed to implement a state machine 40 indicative of one or more states of the light output aperture 20 relative to the sheath end 16. For example, the electronic processor 36 is programmed to determine an inside state of the light output aperture 20 indicative of whether the light output aperture is inside of the containment sheath 14 (e.g., based on the detected passage events by the aperture marker 24 and the sheath opening marker 26 detecting the light output aperture 20 being within the containment sheath 14). For example, if it is known that at time to the light output aperture 20 is inside the containment sheath 14 (e.g., if this is the state when the catheter is inserted into the blood vessel), then the state machine 40 can beset to an inside state of Tip-State=inside safe zone.

The electronic processor 36 is programmed to update a state from the inside state to an outside state after determining that the light output aperture 20 is outside of the containment sheath 14 (e.g., the sensors 24, 26 have detected a passage event that the light outpour aperture 20 has extended beyond the sheath opening 18), i.e., Tip-State-outside safe zone. In some examples, the electronic processor 36 is programmed to further update the state of the light output aperture 20 from the outside state back to the inside state by determining whether the aperture marker 24 no longer extends past the sheath opening marker 26. For example, at a time t₁ that is later than the initial time t₀, the sensors 24, 26 can detect an event indicating the light output aperture 20 passes through the sheath opening 18. This detected event can be assumed to be due to the light output aperture 20 being extended beyond the sheath opening 18, so that the state machine 40 can be updated from Tip-State=inside safe zone to Tip-State=outside safe zone. In another example, at a time t₂ later than t₁ the sensors 24, 26 again detects an event indicating passage of the light output aperture 20 through the sheath opening 18. This can be assumed to be a retraction operation and the state can be updated from Tip-State=outside safe zone to be switched back to Tip-State=inside safe zone. In other words, every time the sensors 24, 26 detect an event indicating the light output aperture 20 passes through the sheath opening 18, the state can be updated to that the variable Tip-state can be switched from either inside to outside or from outside to inside.

In some embodiments, when the device 10 includes the backside proximity sensor 28, the state machine 40 can include a third state Tip-State-overwithdrawn. Each time the backside proximity sensor 28 detects an event of the light output aperture 20, the state machine 40 can be updated from Tip-State=inside safe zone to Tip-State=overwithdrawn, or from Tip-State=overwithdrawn to Tip-State=inside safe zone. It will be appreciated that there will never be a transition of the state machine 40 in which the state is updated from Tip-State=overwithdrawn to Tip-State=outside safe zone (or vice versa), as the light output aperture 20 would first have to pass through the sheath opening marker 26 which should switch the state machine 40 from Tip-State=outside safe zone to Tip-State=inside safe zone. In some embodiments, the state of light output aperture 20 can be displayed on the display device 37 (e.g., “inside”, “open”, “overwithdrawn”, and so forth).

The device 10 is configured to operate in a saline mode in which saline flows through the containment sheath 14, and in a contrast mode in which contrast medium flows through the containment catheter 14. The saline is typically flowed during operation in which the light output at the aperture 20 directly ablates the lesion. In this saline mode, the aperture 20 is extended outside the sheath opening 16. The contrast medium is used in contrast mode to provide light absorption and response effective to produce light induced pressure waves (LIPW) thereby providing a different removal mechanism, one that is particularly well suited for removing calcified deposits that have low absorption for the light. In this mode, the light output aperture 20 should be withdrawn into the safe zone inside the sheath 14. In addition to tracking the state of the light output aperture 20 relative to the sheath opening 18 using the state machine 40, the electronic processor 36 is programmed to also track whether the catheter 12 is in the saline mode or the contrast mode, for example by way of a manual input, an optical sensor, by direct control of valves controlling the saline and contrast medium flows, or so forth. If the electronic processor 36 determines that (i) the catheter 12 is in the contrast mode and (ii) the state of the light output aperture is in the outside state, then the electronic processor 36 is configured to control the laser coupler 34 to cease supplying optical energy to the at least one optical fiber 18 (i.e., so that the ablation process is halted).

In some embodiments, in lieu of the electronic processor 36 controlling the supply of energy to the optical fiber(s) 18, the device 10 includes a device controller 42 that is configured to automatically turn off the laser coupler 34 operating to inject light into the at least one optical fiber 18 of the catheter in response to a safety interlock condition in which the device 10 is operating in contrast mode and the safety sensor 22 detects that the light output aperture 20 is not in the safe zone inside the containment sheath 14. In another suitable approach, the laser itself may be turned off in response to the safety interlock condition. The safety interlock condition can include the electronic processor 36 determining that (i) the catheter 12 is in the contrast mode and (ii) the state of the light output aperture is in the outside state.

FIG. 4 shows a front view of the device 10. Specifically, FIG. 4 shows an end view of the inner catheter 12 (i.e., looking “straight-on” at the light outlet aperture 20). FIG. 4 shows the spatial relationship between the light outlet aperture 20 (which in this example is the end of a fiber optical bundle), an optional guide wire lumen 50 through which a guide wire used for guiding the catheter extends, and an illustrative additional lumen 52. If the aperture marker 24 is an active marker, then for example the additional lumen 52 may provide the conduit for electrical wires running to the active aperture marker 24. In this nonlimiting illustrative embodiment, the aperture 20 is arranged eccentrically relative to the center axis of the inner catheter 12.

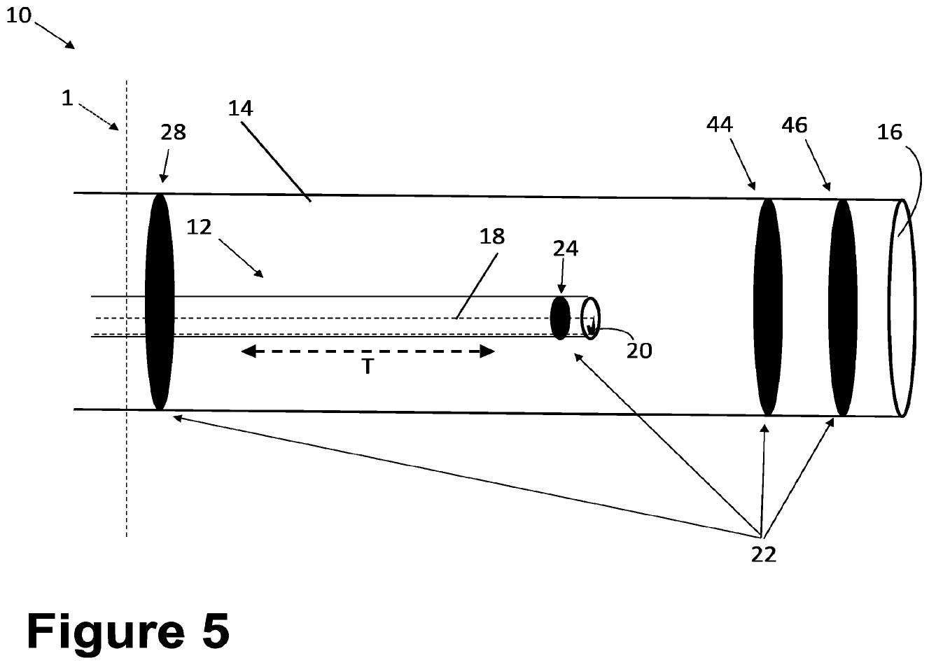

FIG. 5 shows another embodiment of the device 10. As shown in FIG. 5, the proximity sensor 22 can include a first sensor 44 and a second sensor 46 disposed adjacent or close to the sheath opening 18. The second sensor 46 is disposed closer to the sheath opening 18 than the first sensor 44. When the catheter 12 (and thus the light output aperture 20) is extended through the containment sheath 14 and out of the sheath opening 18, this passage event is first detected by the first sensor 44, and then the second sensor 46. Conversely, when the extended light output aperture 20 is withdrawn into the containment sheath 14, this passage event is first detected by the second sensor 46, and then by the first sensor 44. Advantageously, the accuracy of the proximity sensor 22 is improved.

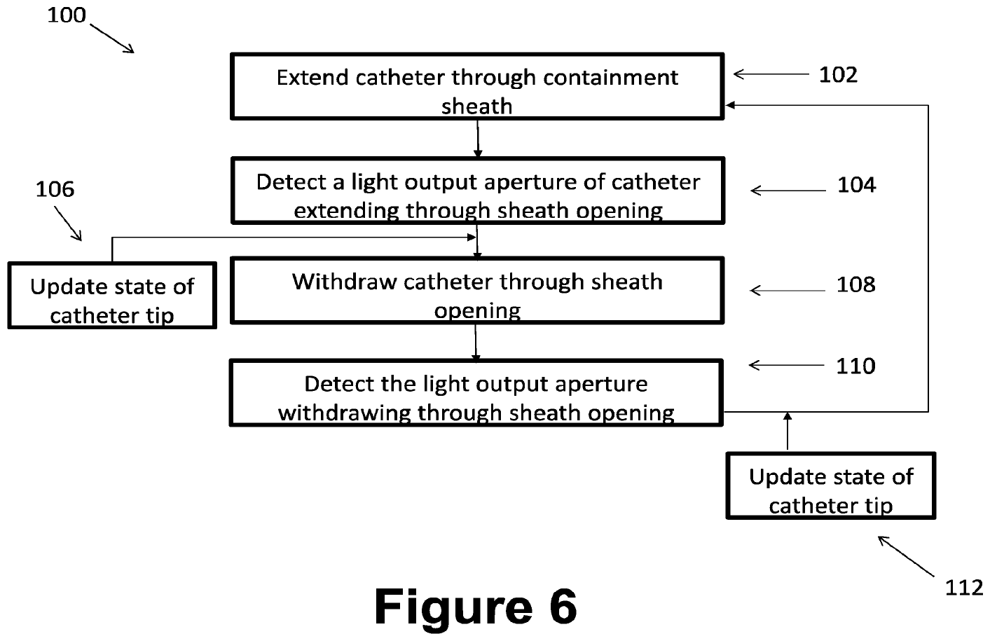

FIG. 6 shows an example of a flowchart showing a vascular therapy method 100 using the device 10. To being the method 100, the device 10 is inserted into an artery or vein of the patient. Energy is supplied to the optical fiber(s) 18 via the laser coupler 34 for ablation of the lesion. At an operation 102, the catheter 12 is extended through the containment sheath 14. At an operation 104, the safety sensor 22 is configured to detect a passage event of the light outlet aperture 20 extending beyond the sheath opening 18. In some embodiments, in an optional operation 106, the state machine 40 is updated to the outside state once the light outlet aperture 20 extends beyond the sheath opening 18. At an operation 108, the catheter 12 is withdrawn through the sheath opening 18 such that the light outlet aperture 20 is within the containment sheath 14. At an operation 110, the safety sensor 22 is configured to detect a passage event of the light outlet aperture 20 extending past the sheath opening 18 and into an interior of the containment sheath 14. In some embodiments, in an optional operation 112, the state machine 40 is updated to the inside state once the light outlet aperture 20 is detected to be within the containment sheath 14. The method 100 can be performed iteratively until a treatment session is over.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A vascular therapy device, comprising:
a catheter including at least one optical fiber with a light output aperture;
a containment sheath having a sheath opening disposed at an end thereof, the catheter disposed inside the containment sheath and movable relative to the containment sheath to extend the light output aperture beyond the sheath opening such that the light output aperture is outside of the containment sheath; and
a safety sensor configured to detect whether the light output aperture is in a safe zone inside the containment sheath.

2. The device of claim 1, wherein the safety sensor includes:
a proximity sensor configured to detect passage events in which the light output aperture passes through the sheath opening; and
an electronic processor configured to detect whether the light output aperture is in the safe zone based at least on the passage events detected by the proximity sensor.

3. The device of claim 2, wherein the proximity sensor includes:
an aperture marker disposed on the catheter; and
a sheath opening marker disposed on the containment sheath;
wherein one of the aperture marker and the sheath opening marker is a passive marker and the other of the aperture marker and the sheath opening marker is an active marker configured to detect proximity of the passive marker.

4. The device of claim 2, wherein the safety sensor further includes:
a backside proximity sensor configured to detect passage events in which the light output aperture passes through an interior boundary of the safe zone wherein the interior boundary is located inside the containment sheath;
wherein the electronic processor is configured to detect whether the light output aperture is in the safe zone further based on the passage events detected by the backside proximity sensor.

5. The device of claim 1, wherein the safety sensor includes:
a radiopaque aperture marker disposed on the catheter;
a radiopaque sheath opening marker disposed on the containment sheath; and
an electronic processor configured to:
receive images from an interventional radiology imaging device that depict the radiopaque aperture marker and the radiopaque sheath opening marker; and
detect whether the light output aperture is in the safe zone based on the received images.

6. The device of claim 1, wherein the device is configured to operate in a saline mode in which saline flows through the containment sheath and in a contrast mode in which contrast medium flows through the containment sheath, the device further comprising:
a device controller configured to automatically turn off a laser operating to inject light into the at least one optical fiber of the catheter in response to a safety interlock condition in which the device is operating in contrast mode and the safety sensor detects that the light output aperture is not in the safe zone inside the containment sheath.

7. The device of claim 2, wherein the electronic processor is programmed to implement a state machine by:
determining an inside state of the light output aperture indicative of whether the light output aperture is inside of the containment sheath;
update the state machine from the inside state to an outside state after determining that the light output aperture is outside of the containment sheath;
determine whether the catheter is in the saline mode or the contrast mode; and
cease supplying optical energy to the at least one optical fiber when (i) the catheter is in the contrast mode and (ii) the state of the light output aperture is in the outside state.

8. The device of claim 7, wherein the electronic processor is further programmed to:
update the state machine of the light output aperture from the outside state to the inside state by determining whether the aperture marker no longer extends past the sheath opening marker.

9. A vascular therapy device, comprising:
a catheter including at least one optical fiber with a light output aperture;
a containment sheath having a sheath opening disposed at an end thereof, the catheter disposed inside the containment sheath and movable relative to the containment sheath to extend the light output aperture beyond the sheath opening such that the light output aperture is outside of the containment sheath;
a proximity sensor configured to detect passage events in which the light output aperture passes through the sheath opening; and
an electronic processor configured to detect whether the light output aperture is in the safe zone based at least on the passage events detected by the proximity sensor.

10. The device of claim 9, wherein the device is configured to operate in a saline mode in which saline flows through the containment sheath and in a contrast mode in which contrast medium flows through the containment sheath, the device further comprising:
a device controller configured to automatically turn off a laser operating to inject light into the at least one optical fiber of the catheter in response to a safety interlock condition in which the device is operating in contrast mode and the proximity sensor detects that the light output aperture is not in the safe zone inside the containment sheath.

11. The device of claim 9, wherein the proximity sensor includes:
an aperture marker disposed on the catheter; and
a sheath opening marker disposed on the containment sheath;
wherein one of the aperture marker and the sheath opening marker is a passive marker and the other of the aperture marker and the sheath opening marker is an active marker configured to detect proximity of the passive marker.

12. The device of claim 9, wherein the electronic processor is programmed to implement a state machine by:
determining an inside state of the light output aperture indicative of whether the light output aperture is inside of the containment sheath;
update the state machine from the inside state to an outside state after determining that the light output aperture is outside of the containment sheath;
determine whether the catheter is in the saline mode or the contrast mode; and
cease supplying optical energy to the at least one optical fiber when (i) the catheter is in the contrast mode and (ii) the state of the light output aperture is in the outside state.

13. The device of claim 12, wherein the electronic processor is further programmed to:
update the state machine of the light output aperture from the outside state to the inside state by determining whether the aperture marker no longer extends past the sheath opening marker.

14. The device of claim 12, wherein the proximity sensor further includes:
a backside proximity sensor configured to detect passage events in which the light output aperture passes through an interior boundary of the safe zone wherein the interior boundary is located inside the containment sheath;
wherein the electronic processor is configured to detect whether the light output aperture is in the safe zone further based on the passage events detected by the backside proximity sensor.

15. A vascular therapy device, comprising:
a catheter including at least one optical fiber with a light output aperture;
a containment sheath having a sheath opening disposed at an end thereof, the catheter disposed inside the containment sheath and movable relative to the containment sheath to extend the light output aperture beyond the sheath opening such that the light output aperture is outside of the containment sheath; and
a safety sensor configured to detect whether the light output aperture is in a safe zone inside the containment sheath;
wherein the device is configured to operate in a saline mode in which saline flows through the containment sheath and in a contrast mode in which contrast medium flows through the containment sheath, the device further comprising:
a device controller configured to automatically turn off a laser operating to inject light into the at least one optical fiber of the catheter in response to a safety interlock condition in which the device is operating in contrast mode and the safety sensor detects that the light output aperture is not in the safe zone inside the containment sheath.

16. The device of claim 15, wherein the safety sensor includes:
a proximity sensor configured to detect passage events in which the light output aperture passes through the sheath opening; and
an electronic processor configured to detect whether the light output aperture is in the safe zone based at least on the passage events detected by the proximity sensor.

17. The device of claim 16, wherein the proximity sensor includes:
an aperture marker disposed on the catheter; and
a sheath opening marker disposed on the containment sheath;
wherein one of the aperture marker and the sheath opening marker is a passive marker and the other of the aperture marker and the sheath opening marker is an active marker configured to detect proximity of the passive marker.

18. The device of claim 16, wherein the safety sensor further includes:
a backside proximity sensor configured to detect passage events in which the light output aperture passes through an interior boundary of the safe zone wherein the interior boundary is located inside the containment sheath;
wherein the electronic processor is configured to detect whether the light output aperture is in the safe zone further based on the passage events detected by the backside proximity sensor.

19. The device of claim 15, wherein the electronic processor is programmed to implement a state machine by:
determining an inside state of the light output aperture indicative of whether the light output aperture is inside of the containment sheath;
update the state machine from the inside state to an outside state after determining that the light output aperture is outside of the containment sheath;
determine whether the catheter is in the saline mode or the contrast mode; and
cease supplying optical energy to the at least one optical fiber when (i) the catheter is in the contrast mode and (ii) the state of the light output aperture is in the outside state.

20. The device of claim 19, wherein the electronic processor further programmed to:
update the state machine of the light output aperture from the outside state to the inside state by determining whether the aperture marker no longer extends past the sheath opening marker.
